(19) United States
(12) Patent Application Publication
(10) Pub. No.: US 2025/0186342 A1
(43) Pub. Date: Jun. 12, 2025

(54) ANTIEMETIC COMBINATIONS

(71) Applicant: SPES PHARMACEUTICALS INC., Plainsboro, NJ (US)

(72) Inventor: Jianwei YU, Plainsboro, NJ (US)

(21) Appl. No.: 18/922,854

(22) Filed: Oct. 22, 2024

Related U.S. Application Data
(63) Continuation of application No. PCT/US2024/043087, filed on Aug. 20, 2024.
(60) Provisional application No. 63/547,250, filed on Nov. 3, 2023.

(51) Int. Cl. A61K 9/08; A61K 9/00; A61K 9/14; A61K 9/19; A61K 31/473; A61K 31/675; A61K 47/02; A61K 47/18; A61K 47/40

(52) U.S. Cl. CPC A61K 9/08; A61K 9/0019; A61K 9/145; A61K 9/146; A61K 9/19; A61K 31/473; A61K 31/675; A61K 47/02; A61K 47/183; A61K 47/40

(57) ABSTRACT

Provided herein are novel ready-to-use formulations containing fosaprepitant and palonosetron, methods of preparing, and methods of using the same.

This application is a continuation of International Application No. PCT/US2024/043087, filed Aug. 20, 2024, which claims the benefit of priority to U.S. Provisional Application No. 63/547,250, filed Nov. 3, 2023, the content of each of which is herein incorporated by reference in its entirety.

This disclosure generally relates to the field of compositions of NK1 receptor antagonists and 5-HT₃ receptor antagonists, methods of preparation, and methods of using the same, for example, in treating or preventing nausea and vomiting.

### BACKGROUND

Various neurokinin-1 (NK1) receptor antagonists have been developed for the treatment of substance P involved diseases such as migraine, depression, anxiety, inflammatory conditions associated with the gastrointestinal tract, and nausea and vomiting, particularly for prevention of chemotherapy induced nausea and vomiting (CINV) (SC Huang, VL Korlipara, Neurokinin-1 receptor antagonists: a comprehensive patent survey, Expert opinion on the therapeutic patents, 2010, 20(8)1019-1045) and postoperative nausea and vomiting.

Fosaprepitant is an NK1 receptor antagonist that is proven to be effective in the treatment of CINV. Fosaprepitant dimeglumine, a prodrug of aprepitant, chemically described as 1-Deoxy-1-(methylamino)-D-glucitol[3-[[(2R,3S)-2-[(1R)-1-[3,5-bis(trifluoromethyl)phenyl]ethoxy]-3-(4-fluorophenyl)-4-morpholinyl]methyl]-2,5-dihydro-5-oxo-1H-1,2,4-triazol-1-yl]phosphonate (2:1) (salt), has the structure:

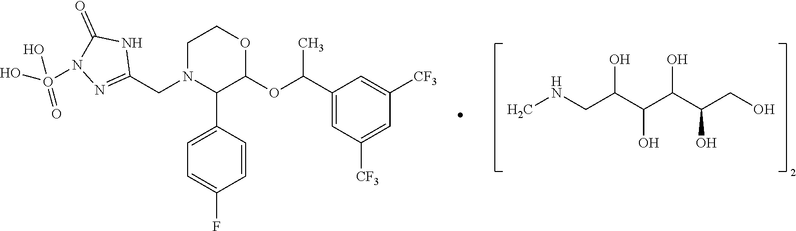

Similar to substance P, 5-hydroxytryptamine-3 (5-HT₃) is a neurotransmitter that acts on the corresponding serotonin receptor and is involved in biological responses including nausea and vomiting (P J Hesketh, P Sanz-Altamira, Aprepitant, dexamethasone, and palonosetron in the prevention of doxorubicin/cyclophosphamide-induced nausea and vomiting, Support Care Cancer (2012) 20:653-656). Palonosetron is a second-generation 5-HT₃ receptor antagonist that differs from older first-generation 5-HT3 receptor antagonists, such as ondansetron, granisetron, dolasetron, and tropisetron, in its prolonged half-life and greater receptor binding affinity. Palonosetron hydrochloride, chemically described as 1H-Benzo[de]isoquinoline-1-one, 2,3,3a,4,5,6-hexahydro-2-[(3S)-1-azabicyclo[2.2.2]octan-3-yl],(S)-hydrochloride, has the structure:

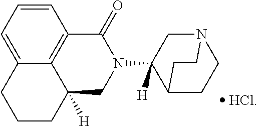 [SMILES: O=C1c2cccc3c2[C@H](CCC3)CN1[C@@H]1CN2CCC1CC2]

Current treatment for both acute and delayed nausea and vomiting includes NK₁ receptor antagonists and 5-HT₃ receptor antagonists to a subject in need thereof. Several efficacy studies of combining the NK₁ receptor antagonist Aprepitant with 5-HT₃ receptor antagonist palonosetron and steroid dexamethasone proved that the combination is generally well-tolerated and provided consistently superior protection against CINV in subjects receiving highly emetogenic cisplatin-based chemotherapy (Weinstein C, et al. Single-dose fosaprepitant for the prevention of chemo-therapy-induced nausea and vomiting associated with moderately emetogenic chemotherapy: results of a randomized, double-blind phase III trial. Ann Oncol. 2016; 27(1):172-8; Kumagai, Hozumi et al. “Efficacy and safety of an increased-dose of dexamethasone in patients receiving fosaprepitant chemotherapy in Japan.” Asian Pacific journal of cancer prevention: APJCP vol. 15,1 (2014): 461-5. doi:10.7314/apjcp.2014.15.1.461). It has been discovered that palonosetron is much more effective in combination with aprepitant than single-dose administration of palonosetron (SM Grunberg, M Dugan, H Muss, et. al, Effectiveness of a single-day three-drug regimen of dexamethasone, palonosetron, and aprepitant for the prevention of acute and delayed nausea and vomiting caused by moderately emetogenic chemotherapy, Support Cancer care 2009, 17(5)589-94).

Therefore, co-formulation or combination of an NK₁ receptor antagonist and a 5-HT₃ receptor antagonist, such as fosaprepitant or aprepitant and palonosetron into a single dosage form shall reduce the number of and/or volume of injections, improve clinical management, and reduce healthcare costs. There is a strong medical demand to develop effective multimodal therapies which gives two or more antiemetic drugs of different modes of actions at the same time in the same dosage form.

Fosaprepitant is a very unstable compound in aqueous solutions and therefore has to be formulated in its dry form, such as lyophilized formulation of Emend for injection. EMEND® for Injection is currently the only approved injection product of fosaprepitant. The injection comprises a sterile, lyophilized powder formulation containing fosaprepitant dimeglumine. Each vial of EMEND® for Injection contains 150 mg of fosaprepitant (equivalent to 245.3 mg of fosaprepitant dimeglumine) and other inactive ingredients, such as edetate disodium and polysorbate 80, etc. The formulation of Emend includes polysorbate 80, a commonly used solubilizing agent for low solubility drug formulation, which is also known to cause hypersensitivity reactions including life-threatening anaphylactic reactions. FOCINVEZ (fosaprepitant injection) is another FDA approved injection of fosaprepitant. Both EMEND and FOCINVEZ are single active drug products.

Palonosetron has been developed for antiemetic injection products, alone or in combination with other antiemetic agent, such as Fosnetupitant, a prodrug of netupitant.

### BRIEF SUMMARY

In various embodiments, the present disclosure generally relates to liquid formulations, such as aqueous solutions, containing both fosaprepitant and palonosetron, which are typically storage stable for an extended period of time. In some embodiments, the liquid formulations can be a ready-to-use formulation, which can be directly used for administration to a subject in need, without further handling and manipulation, such as reconstitution and dilution. In some embodiments, the liquid formulations can also be a ready-to-dilute formulation, which can be diluted and then used for administration to a subject in need.

The present disclosure is based in part on the unexpected discovery that fosaprepitant and palonosetron can be formulated in a ready-to-use or ready-to-dilute aqueous solution with good storage stability. This is surprising because it was known that fosaprepitant and palonosetron typically are stable and used under different pHs, with fosaprepitant in a basic pH and palonosetron in an acidic pH. As shown in the Examples section, exemplary formulations containing both fosaprepitant and palonosetron, were found to be physically and chemically stable when stored at room temperature (25° C.+/−2° C.) for 6 months. These exemplary formulations are also characterized in that (i) the formulations have a limited number of excipients, (ii) the formulations do not use surfactants such as polysorbate, which may cause potential allergic or other adverse reactions, and (iii) the formulations do not use organic solvents. With the enhanced storage stability, this disclosure shows that a ready-to-use aqueous formulation containing both fosaprepitant and palonosetron can be prepared, which can provide significant benefits to patients in need of such treatment. For example, non-limiting benefits of using the ready-to-use aqueous formulations herein include the following: (1) it reduces the number and volume of injections, reduce discomfort and therefore improve patient compliance; (2) it reduces hypersensitivity and other adverse reaction that may be caused by polysorbate 80 or other surfactant or other egg or soy products in a formulation; (3) it prevents hydrolysis of fosaprepitant and/or prevents aprepitant from crystallization and precipitation, thereby keeping the active ingredients in solution state without the need to use an organic non-aqueous solvents or co-solvents; and (4) it eliminates nursing time associated with lyophilized product reconstitution preparation and dilution for the administration of intravenous injections and to enable overall cost savings (see e.g., P Van der Linden, J Douchamps, C Schmitt, et. al., Ready-to-Use Injection Preparations versus Conventional Reconstituted Admixtures. Pharmaco Economics, 20(8)529-536).

The disclosure herein also solves many of the problems encountered by others. For example, the aqueous pharmaceutical compositions herein do not have one or more drawbacks associated with those in US2019/0358249A1, which pertains to powder for injection formulation combining fosaprepitant dimeglumine and palonosetron hydrochloride. Several disadvantages of US2019/0358249A1 are: (1) The formulation includes polysorbate 80 which causes hypersensitive reactions, (2) requires reconstitution (3) high cost due to the freeze-drying process. These disadvantages can be readily resolved by embodiments of this disclosure.

Patent CN102755338 relates lyophilized injectable combines fosaprepitant dimeglumine and palonosetron hydrochloride into a single formulation. The formulation contains polysorbate 80 and requires further reconstitution. These disadvantages can be readily resolved by embodiments of this disclosure.

Patent Application US2015/0320866A1 and US2017/0232107A1 relate to sustained release formulation combines both NK₁ receptor antagonist and 5-HT₃ receptor antagonist, in one formulation which is a semi-solid delivery vehicle includes polyorthoester excipient, for subcutaneous injection. Embodiments of this disclosure do not have the disadvantages of using such semi-solid delivery system or polyorthoester excipient.

Patent U.S. Pat. No. 9,913,853 and US202/0264A1 both relate to stable pharmaceutical compositions of fosaprepitant or a salt thereof in the form of ready-to-use or ready-to-dilute compositions suitable for parenteral administration and specifically contains human albumin as a stabilizer and/or in various organic solvents. Embodiments of this disclosure do not use such stabilizer or organic solvent, which adds complexity to the formulation and can cause potential side effects.

In some embodiments, the present disclosure provides the following exemplary embodiments [1]-[31]:

[1] An aqueous solution suitable for parenteral injection comprising:
(1) fosaprepitant, or a pharmaceutically acceptable salt thereof;
(2) palonosetron, or a pharmaceutically acceptable salt thereof;
(3) a cyclodextrin;
(4) a water-soluble antioxidant; and
(5) water;
wherein the aqueous solution comprises water in an amount of greater than 50% by weight, e.g., about 60%, about 70%, about 80%, about 85%, about 90%, or any ranges or values between the recited values, such as about 60-85%, about 60-95%, about 70-85%, about 70-90%, etc.;
wherein the concentration of fosaprepitant in the aqueous solution ranges from about 1.6 mg/mL to about 16.4 mg/mL;
the concentration of palonosetron in the aqueous solution ranges from about 1 μg/mL to about 16.7 μg/mL;
the weight ratio of fosaprepitant to the cyclodextrin ranges from about 1:10 to about 1:255; and
the pH of the aqueous solution ranges from about 9 to about 12,
wherein the concentration of fosaprepitant or weight ratio of fosaprepitant to the cyclodextrin is calculated based on equivalent weight to fosaprepitant dimeglumine salt; and the concentration of palonosetron is calculated based on equivalent weight to palonosetron hydrochloride salt.

[2] The aqueous solution of [1], wherein the water-soluble antioxidant is a sulfur containing antioxidant, such as an amino acid, such as methionine, or monothioglycerol, or sodium metabisulfite, etc.

[3] The aqueous solution of [1], wherein the water-soluble antioxidant is methionine.

[4] The aqueous solution of any of [1]-[3], wherein the water-soluble antioxidant is present in the aqueous solution at a concentration ranging from about 0.1 mg/ml to about 30 mg/ml, such as about 0.5 mg/ml, about 1 mg/ml, about 1.5 mg/ml, about 2 mg/ml, about 2.5 mg/ml, about 5 mg/ml, about 10 mg/ml, about 15 mg/ml, about 20 mg/ml, about 30 mg/ml, or any ranges or values between the recited values, such as about 1.5-2.5 mg/ml, about 2-5 mg/ml, etc.

[5] The aqueous solution of any of [1]-[4], wherein the cyclodextrin is a water-soluble derivative of beta-cyclodextrin.

[6] The aqueous solution of any of [1]-[4], wherein the cyclodextrin is sulfobutyl ether beta-cyclodextrin, preferably, a sulfobutyl ether-β-cyclodextrin, or a sodium salt thereof, having an average degree of substitution of about 3-10, such as about 4.5-7.5 or about 6-7.1.

[7] The aqueous solution of any of [1]-[6], wherein the cyclodextrin is present in the aqueous solution at a concentration ranging from about 50 mg/ml to about 300 mg/ml, such as about 50 mg/ml, about 60 mg/ml, about 70 mg/ml, about 80 mg/ml, about 90 mg/ml, about 100 mg/ml, about 120 mg/ml, about 140 mg/ml, about 160 mg/ml, about 180 mg/ml, about 200 mg/ml, about 250 mg/ml, about 300 mg/ml, or any ranges or values between the recited values, such as about 60-200 mg/ml, about 80-180 mg/ml, about 64 mg/ml, etc.

[8] The aqueous solution of any of [1]-[7], wherein the concentration of fosaprepitant in the aqueous solution ranges from about 1.6 mg/mL to about 9.8 mg/mL.

[9] The aqueous solution of any of [1]-[8], wherein the concentration of palonosetron in the aqueous solution ranges from about 1 μg/mL to about 11 μg/mL.

[10] The aqueous solution of any of [1]-[9], wherein the weight ratio of fosaprepitant to the cyclodextrin ranges from about 1:13 to about 1:255, such as about 1:20, about 1:25, about 1:30, about 1:35, about 1:40, about 1:50, about 1:75, about 1:100, about 1:150, about 1:200, about 1:250, or any ranges or values between the recited values, such as about 1:20 to about 1:50, about 1:30 to about 1:40; about 1:25 to about 1:100, etc.

[11] The aqueous solution of any of [1]-[10], wherein the pH of the aqueous solution is about 9 to about 11, such as about 9.5, about 10, about 10.5, about 11, or any ranges or values between the recited values, such as about 10-11.

[12] The aqueous solution of any of [1]-[11], wherein the aqueous solution is free of or substantially free of a surfactant.

[13] The aqueous solution of any of [1]-[11], wherein the aqueous solution is free of or substantially free of a surfactant containing a polyoxyethylene unit.

[14] The aqueous solution of any of [1]-[11], wherein the aqueous solution is free of or substantially free of a polysorbate.

[15] The aqueous solution of any of [1]-[14], wherein the aqueous solution is free of or substantially free of a water-soluble polymer or protein.

[16] The aqueous solution of any of [1]-[15], wherein the aqueous solution is free of or substantially free of a buffer agent.

[17] The aqueous solution of any of [1]-[16], wherein the aqueous solution further comprises a chelating agent.

[18] The aqueous solution of [17], wherein chelating agent is EDTA.

[19] The aqueous solution of any of [1]-[18], wherein the aqueous solution has a total number of two to five (2-5) inactive ingredients, not considering water and pH adjusting agent(s).

[20] The aqueous solution of [19], wherein the inactive ingredients in the aqueous solution, not considering water and pH adjusting agent(s), comprise or consist of the cyclodextrin, the water-soluble antioxidant, and a chelating agent.

[21] The aqueous solution of any of [1]-[20], wherein the fosaprepitant and palonosetron, or pharmaceutically acceptable salts thereof, are the only active ingredients in the aqueous solution.

[22] The aqueous solution of any of [1]-[21], which is free of visible precipitation.

[23] The aqueous solution of any of [1]-[22], characterized in that when stored at room temperature (25° C.+/−2° C.) for 6 months, the aqueous solution is characterized by one or more (e.g., two or more, three or more, four or more, or all) of the following: (1) the amount of aprepitant in the aqueous solution is less than 5% by weight of the weight of fosaprepitant; (2) the impurity having a relative retention time of 0.65 to fosaprepitant is less than 0.1% by weight of the weight of fosaprepitant; (3) the impurity having a relative retention time of 0.91 to fosaprepitant is less than 0.1% by weight of the weight of fosaprepitant; (4) the impurity having a relative retention time of 2.32 to fosaprepitant is less than 0.1% by weight of the weight of fosaprepitant; (5) the impurity having a relative retention time of 1.06 to palonosetron is less than 0.1% by weight of the weight of palonosetron; and (6) the aqueous solution remains a clear solution free of visible precipitation.

[24] The aqueous solution of any of [1]-[23], which is a ready-to-use formulation.

[25] The aqueous solution of any of [1]-[24], in a unit dosage form.

[26] The aqueous solution of any of [1]-[25], in a multidose dosage form.

[27]A lyophilized powder made from the aqueous solution of any of [1]-[26].

[28]A method of preparing the aqueous solution of any one of [1]-[26], the method comprising:
(a) dissolving the fosaprepitant or pharmaceutically acceptable salt in water to form a first solution;
(b) mixing the cyclodextrin with the first solution to provide a second solution;
(c) mixing the palonosetron or pharmaceutically acceptable salt thereof, water-soluble antioxidant, and optional other ingredients to the second solution to form a third solution; and
(d) optionally adjusting the pH of the third solution, thereby forming the aqueous solution.

[29]A method of treating nausea and/or vomiting, comprising administering to a subject in need the aqueous solution of any of [1]-[26].

[30] The method of [29], wherein the administering is by parenteral injection.

[31] The method of [29] or [30], wherein the nausea and/or vomiting is a chemotherapy, a radiation therapy, or a surgical procedure induced nausea and/or vomiting.

It is to be understood that both the foregoing summary and the following detailed description are exemplary and explanatory only, and are not restrictive of the invention herein.

### DETAILED DESCRIPTION

In a broad aspect, the present disclosure provides a liquid composition of Fosaprepitant or a pharmaceutically acceptable salt thereof, and a 5-HT3 receptor antagonist or a pharmaceutically acceptable salt thereof, particularly, palonosetron or a pharmaceutically acceptable salt thereof. The liquid composition is typically a ready-to-use aqueous composition, suitable for parenteral administration, such as intravenous injection (including bolus injection and infusion). The liquid composition herein can have various uses for treating disorders known to be treated with fosaprepitant and 5-HT3 receptor antagonists, such as for treating nausea and/or vomiting induced by chemotherapy. As used herein, a ready-to-use formulation means a formulation that can be directly administered without further handling, such as reconstituting or dilution. For example, a ready-to-use formulation may be included in a pre-filled syringe, which can be directly used to inject the formulation to a patient in need. In some embodiments, the liquid composition herein can also be a ready-to-dilute aqueous composition, which means that the liquid can be administered to a patient in need after proper dilution, for example, with an appropriate infusion solution, such as a saline solution. The liquid composition is typically storage stable, e.g., as defined herein.

In some embodiments, the present disclosure provides an aqueous pharmaceutical composition comprising: (1) fosaprepitant, or a pharmaceutically acceptable salt thereof; (2) palonosetron, or a pharmaceutically acceptable salt thereof; (3) a cyclodextrin; (4) a water-soluble antioxidant; and (5) water. Typically, the aqueous pharmaceutical composition is an aqueous solution suitable for parenteral injection, such as intravenous injection. In some embodiments, the aqueous pharmaceutical composition comprises water in an amount greater than 50% by weight. For example, in some embodiments, the aqueous pharmaceutical composition comprises water, by weight percentage, in an amount of about 60%, about 70%, about 80%, about 85%, about 90%, or any ranges or values between the recited values, such as about 60-85%, about 60-95%, about 70-85%, about 70-90%, etc.

Typically, the aqueous pharmaceutical composition comprises fosaprepitant in a concentration ranging from about 1.6 mg/mL to about 16.4 mg/mL. In some embodiments, the fosaprepitant is in a concentration ranging from about 1.6 mg/mL to about 9.8 mg/mL. As used herein, when referring to concentrations of fosaprepitant, it should be understood that the concentration is calculated based on equivalent weight to fosaprepitant dimeglumine salt. For example, when an aqueous pharmaceutical composition herein is prepared with 0.1 mmol of fosaprepitant or 0.1 mmol of a pharmaceutically acceptable salt of fosaprepitant, the concentration of fosaprepitant in the aqueous pharmaceutical composition should be equal to the weight of 0.1 mmol of fosaprepitant dimeglumine salt divided by the volume of the aqueous pharmaceutical composition. The same applies when referring to weight or weight ratio of fosaprepitant in the aqueous pharmaceutical composition herein.

The aqueous pharmaceutical composition generally comprises palonosetron in a concentration ranging from about 1 μg/mL to about 16.7 μg/mL. In some embodiments, the palonosetron is in a concentration ranging from about 1 μg/mL to about 11 μg/mL. As used herein, when referring to concentrations of palonosetron, it should be understood that the concentration is calculated based on equivalent weight to palonosetron hydrochloride salt. For example, when an aqueous pharmaceutical composition herein is prepared with 0.1 mmol of palonosetron or 0.1 mmol of a pharmaceutically acceptable salt of palonosetron, the concentration of palonosetron in the aqueous pharmaceutical composition should be equal to the weight of 0.1 mmol of palonosetron hydrochloride divided by the volume of the aqueous pharmaceutical composition.

The weight ratio of the fosaprepitant to the cyclodextrin in the aqueous pharmaceutical composition herein typically ranges from about 1:10 to about 1:255. For weight ratio calculations, the amount of fosaprepitant is as discussed above, i.e., should be based on equivalent weight of fosaprepitant dimeglumine salt. When the cyclodextrin contains an acidic group, the amount of cyclodextrin used for the weight ratio calculation should be based on the equivalent weight of its sodium salt form of the acidic group. In some embodiments, the weight ratio of fosaprepitant to the cyclodextrin ranges from about 1:13 to about 1:255, such as about 1:20, about 1:25, about 1:30, about 1:35, about 1:40, about 1:50, about 1:75, about 1:100, about 1:150, about 1:200, about 1:250, or any ranges or values between the recited values, such as about 1:20 to about 1:50, about 1:30 to about 1:40; about 1:25 to about 1:100, etc.

It was discovered that the pH of the aqueous pharmaceutical composition can have an impact on its stability. Typically, the pH of the aqueous pharmaceutical composition herein is about 9 to about 12, such as about 9, about 9.5, about 10, about 10.5, about 11, about 11.5, or about 12, or any ranges or values between the recited values, such as about 9-11, 9.5-12, about 10-11, etc. The term “about” as used herein in connection with a pH value should be understood as within ±0.3 units, so a pH of about 10 includes a pH value of 9.7-10.3.

The water-soluble antioxidant in the aqueous pharmaceutical composition herein is typically a sulfur containing antioxidant. For example, in some embodiments, the water-soluble antioxidant is a sulfur containing amino acid, such as methionine. In some embodiments, the water-soluble antioxidant can also be monothioglycerol. In some embodiments, the water-soluble antioxidant can also be sodium metabisulfite. In some preferred embodiments, the water-soluble antioxidant in the aqueous pharmaceutical composition herein is methionine. As used herein, a “water-soluble” ingredient means that at the desired/required concentration, the ingredient can be fully dissolved in the aqueous pharmaceutical composition herein. Typically, a “water-soluble” ingredient herein has a solubility in water of at least 30 mg/ml, preferably, at least 50 mg/ml.

The water-soluble antioxidant in the aqueous pharmaceutical composition herein is preferably at a concentration ranging from about 0.1 mg/ml to about 30 mg/ml, such as about 0.5 mg/ml, about 1 mg/ml, about 1.5 mg/ml, about 2 mg/ml, about 2.5 mg/ml, about 5 mg/ml, about 10 mg/ml, about 15 mg/ml, about 20 mg/ml, about 30 mg/ml, or any ranges or values between the recited values, such as about 1.5-2.5 mg/ml, about 2-5 mg/ml, etc.

The cyclodextrin in the aqueous pharmaceutical composition herein is typically a water-soluble cyclodextrin.

Cyclodextrins (CDs) are groups of cyclic oligosaccharides which have been shown to be useful for formulation various drugs. CDs are cyclic oligosaccharides composed of several D-glucose units linked by α-(1,4) bonds. This cyclic configuration provides a hydrophobic internal cavity and gives the CDs a truncated cone shape. Many hydroxyl groups are situated on the edges of the ring which make the CDs both lipophilic and soluble in water.

The terms “cyclodextrin” or “CD” unless otherwise specified herein, refer generally to a parent or derivatized cyclic oligosaccharide containing a variable number of (α-1,4)-linked D-glucopyranoside units. Each cyclodextrin glucopyranoside subunit has secondary hydroxyl groups at the 2 and 3 positions and a primary hydroxyl group at the 6-position. The terms “parent”, “underivatized”, or “inert”, cyclodextrin refer to a cyclodextrin containing D-glucopyranoside units having the basic formula C6H1206 and a glucose structure without any additional chemical substitutions (e.g., α-cyclodextrin consisting of 6 D-glucopyranoside units, a β-cyclodextrin consisting of 7 D-glucopyranoside units, and a γ-cyclodextrin consisting of 8 D-glucopyranoside units). The physical and chemical properties of a parent cyclodextrin can be modified by derivatizing the hydroxyl groups with other functional groups.

Unless otherwise specified, in embodiments, the aqueous pharmaceutical composition herein (e.g., any of the aqueous solution described in [1]-[26] in the Brief Summary section herein) can comprise a cyclodextrin derivative of the following formula:

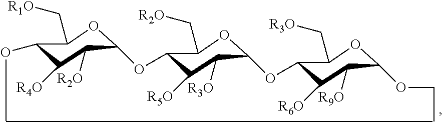

wherein: n is 4, 5, or 6;
wherein R₁, R₂, R₃, R₄, R₅, R₆, R₇, R₈, and R₉ are each, independently, —H, a straight chain or branched C₁-C₈-alkylene group, or an optionally substituted straight-chain or branched C₁-C₆ group, wherein at least one of R₁, R₂, R₃, R₄, R₅, R₆, R₇, R₈ and R₉ is a straight-chain or branched C₁-C₈-alkylene (e.g., C₁-C₈-(alkylene)-SO₃⁻ group).

Unless otherwise specified, in embodiments, the aqueous pharmaceutical composition herein (e.g., any of the aqueous solution described in [1]-[26] in the Brief Summary section herein) can comprise a cyclodextrin derivative of the following formula:

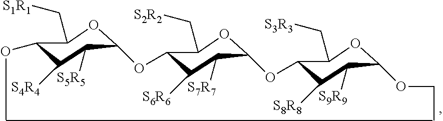

wherein: n is 4, 5, or 6;
wherein R1, R2, R3, R4, R5, R6, R7, R8, and R9 are each, independently, —O— or a —O—(C₂-C₆ alkylene)-SO₃⁻ group; wherein at least one of R1 and R2 is independently a —O—(C₂-C₆ alkylene)-SO₃⁻ group; and S1, S2, S3, S4, S5, S6, S7, S8, and S9 are each, independently, a pharmaceutically acceptable cation. In further embodiments, the pharmaceutically acceptable cation is selected from: an alkali metal such as Li+, Na+, or K+; an alkaline earth metal such as Ca⁺², or Mg⁺² and ammonium ions and amine cations such as the cations of (C1-C6)-alkylamines, piperidine, pyrazine, (C1-C6)-alkanolamine and (C4-C8)-cycloalkanolamine. In some embodiments, at least one of R1 and R2 is independently a —O—(C2-C6 alkylene)-SO₃⁻ group that is a —O—(CH₂)ₘSO3— group, wherein m is 2 to 6, preferably 2 to 4, (e.g., —O—CH2CH2CH2SO3⁻ or —O—CH2CH2CH2CH2SO3⁻); and S₁, S₂, S₃, S₄, S₅, S₆, S₇, S₈, and S₉ are each, independently, H or a pharmaceutically cation which includes for example, alkali metals (e.g., Li⁺, Na⁺, K⁺) alkaline earth metals (e.g., Ca⁺², Mg⁺²), ammonium ions and amine cations such as the cations of (C1-C6)-alkylamines, piperidine, pyrazine, (C₁-C₆)-alkanol-amine and (C₄-C₈)-cycloalkanolamine.

Unless otherwise specified, in embodiments, the aqueous pharmaceutical composition herein (e.g., any of the aqueous solution described in [1]-[26] in the Brief Summary section herein) can comprise a cyclodextrin derivative of the following formula:

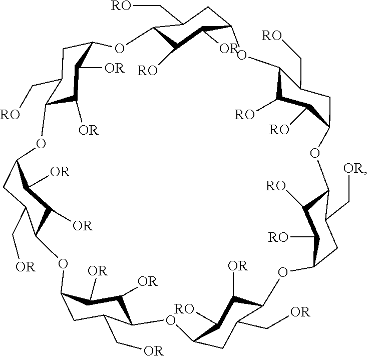

wherein R is:
(a) (H)₂₁₋ₓ or (—(CH₂)₄—SO₃Na)x, and x=1.0-10.0, 1.0-5.0, 6.0-7.0, or 8.0-10.0;
(b) (H)₂₁₋ₓ or (—(CH₂CH(OH)CH₃)x, and x=1.0-10.0, 1.0-5.0, 6.0-7.0, or 8.0-10.0;
(c) (H)₂₁₋ₓ or (sulfoalkyl ethers)x, and x=1.0-10.0, 1.0-5.0, 6.0-7.0, or 8.0-10.0; or
(d) (H)₂₁₋ₓ or (—(CH₂)₄—SO₃Na)x, and x=1.0-10.0, 1.0-5.0, 6.0-7.0, or 8.0-10.0.

Unless otherwise specified, in embodiments, the aqueous pharmaceutical composition herein (e.g., any of the aqueous solution described in [1]-[26] in the Brief Summary section herein) can comprise a sulfoalkyl ether cyclodextrin, such as sulfoalkyl ether beta-cyclodextrin. In some embodiments, the aqueous pharmaceutical composition herein can comprise a hydroxyalkyl ether cyclodextrin, such as hydroxyalkyl ether beta-cyclodextrin. Non-limiting useful cyclodextrins for the aqueous pharmaceutical compositions herein can include any of those known in the art, such as those commercially available and any of those described in U.S. Pat. Nos. 6,133,248, 5,874,418, 6,046,177, 5,376,645, 5,134,127, 7,034,013, 6,869,939, 6,153,746, and 10,117,951; and Intl. Appl. Publ. No. WO 2005/117911, and WO 2009/134347, the content of each of which is herein incorporated by reference in its entirety.

For example, unless otherwise specified, in embodiments, the aqueous pharmaceutical composition herein (e.g., any of the aqueous solution described in [1]-[26] in the Brief Summary section herein) can comprise a sulfobutyl ether-β-cyclodextrin having an average degree of substitution of about 3-10, such as about 4.5-7.5 or about 6-7.1. The sulfobutyl ether-β-cyclodextrin herein is typically in a salt form, such as a sodium salt. One of such sulfobutyl ether beta-cyclodextrin is commercially available as CAPTISOL® (CyDex Pharmaceuticals, Inc., Lenexa, Kans.), which has the following chemical structure:

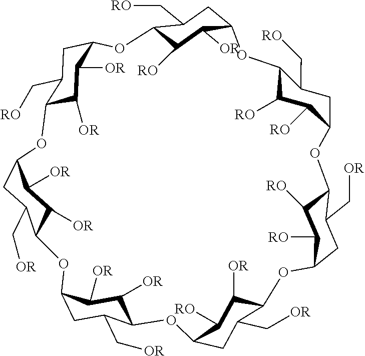

wherein R is (—H)₂₁₋ₙ or (CH2CH2CH2CH2SO3⁻Na⁺)ₙ, and n is 6-7.1.

Unless otherwise specified, in embodiments, the aqueous pharmaceutical composition herein (e.g., any of the aqueous solution described in [1]-[26] in the Brief Summary section herein) can comprise a hydroxypropyl β-cyclodextrin, e.g., those having an average degree of substitution of about 1-10, such as about 2-8, such as about 4.2-6.3, such as about 4.5. Various hydroxypropyl β-cyclodextrins are also known in the art and commercially available, which can be used for the aqueous pharmaceutical compositions herein.

In some preferred embodiments, the cyclodextrin in the aqueous pharmaceutical composition herein is sulfobutyl ether beta-cyclodextrin, preferably, a sulfobutyl ether-β-cyclodextrin having an average degree of substitution of about 3-10, such as about 4.5-7.5 or about 6-7.1, or a sodium salt thereof.

The cyclodextrin is typically presented in the aqueous pharmaceutical composition herein in a concentration ranging from about 50 mg/ml to about 300 mg/ml, such as about 50 mg/ml, about 60 mg/ml, about 70 mg/ml, about 80 mg/ml, about 90 mg/ml, about 100 mg/ml, about 120 mg/ml, about 140 mg/ml, about 160 mg/ml, about 180 mg/ml, about 200 mg/ml, about 250 mg/ml, about 300 mg/ml, or any ranges or values between the recited values, such as about 60-200 mg/ml, about 80-180 mg/ml, about 64 mg/ml, etc. The concentration of the cyclodextrin should be calculated based on equivalent weight of the sodium salt of the cyclodextrin divided by the volume of the aqueous pharmaceutical composition, when the cyclodextrin has an acidic group, i.e., the acid group or salt thereof is converted into sodium salt for the calculation.

In some embodiments, the aqueous pharmaceutical composition herein can be characterized as being free of or substantially free of a surfactant.

In some embodiments, the aqueous pharmaceutical composition herein can be characterized as being free of or substantially free of a surfactant containing a polyoxyethylene unit.

In some embodiments, the aqueous pharmaceutical composition herein can be characterized as being free of or substantially free of a polysorbate.

In some embodiments, the aqueous pharmaceutical composition herein can be characterized as being free of or substantially free of polysorbate 80.

In some embodiments, the aqueous pharmaceutical composition herein can be characterized as being free of or substantially free of a water-soluble polymer or protein.

In some embodiments, the aqueous pharmaceutical composition herein can be characterized as being free of or substantially free of albumin.

In some embodiments, the aqueous pharmaceutical composition herein can be characterized as being free of or substantially free of a buffer agent.

Unless otherwise specified, in embodiments, the aqueous pharmaceutical composition herein (e.g., any of the aqueous solution described in [1]-[26] in the Brief Summary section herein) can also include optional other ingredients, such as a preservative, a chelating agent, an osmotic agent, a buffer, or a combination thereof.

For example, in some embodiments, the aqueous pharmaceutical composition herein can also include a chelating agent. Suitable chelating agents are not particularly limited so long as they are pharmaceutically acceptable, such as those suitable for parenteral administration. For example, in some embodiments, the chelating agent can be selected from the group consisting of: ethylene diamine tetraacetate (EDTA), methylglycinediacetic acid or N,N′-bis(carboxymethyl)alanine (MGDA), ethylene glycol tetraacetic acid (EGTA), (1,4,7,10-tetraazacyclododecane-1,4,7,10-tetraacetic acid) (DOTA), diethylene triamine penta acetic acid (DTPA), diethylene triamine penta methylene phosphonic acid (DTPMP), (1,4,10,13-tetraoxa-7,16-diazacyclooctadecane-7) (ODDA), (1,7,13-triaza-4,10,16-trioxacyclooctadecane-N,N′,N″-triacetate) (TTTA), (tetraethyleneglycol-1,5,9-triazacyclododecane-N,N′,N″, tris(methylene phosphonic acid) (DOTRP), and mixtures thereof. In some specific embodiments, the chelating agent can be ethylene diamine tetraacetate (EDTA), which is typically used in a salt form, such as EDTA disodium salt. In some embodiments, the aqueous pharmaceutical composition herein can also be free of a chelating agent as described herein. For example, in some embodiments, the aqueous pharmaceutical composition herein can be free of EDTA, MGDA, EGTA, DOTA, DTPA, DTPMP, ODDA, TTTA, DOTRP, and combinations thereof.

When present, the chelating agent such as EDTA is typically included in the aqueous pharmaceutical composition herein (e.g., any of the aqueous solution described in [1]-[26] in the Brief Summary section herein) at a concentration of about 0.01 mg/mL to about 10 mg/mL, such as about 0.01 mg/mL, about 0.1 mg/mL, about 0.5 mg/mL, about 1 mg/mL, about 2 mg/mL, about 5 mg/mL, about 10 mg/mL, or any ranges between the recited values. In some preferred embodiments, when present, the chelating agent (such as EDTA) can be included in the aqueous pharmaceutical composition herein (e.g., any of the aqueous solution described in [1]-[26] in the Brief Summary section herein) at a concentration of about 0.1 mg/mL to about 1 mg/mL. The concentration of EDTA can be calculated by dividing the equivalent weight of EDTA disodium salt by the volume of the aqueous pharmaceutical composition.

In some embodiments, the aqueous pharmaceutical composition herein is characterized as having only a small number of excipients or inactive ingredients. Without wishing to be bound by theories, it is believed that by using a small number of excipients, the chances of the aqueous pharmaceutical composition inducing an allergic or other adverse reaction are also reduced. In some embodiments, this can also lead to easier manufacturing.

In some embodiments, the aqueous pharmaceutical composition herein (e.g., any of the aqueous solution described in [1]-[26] in the Brief Summary section herein) has a total number of two to five (2-5) inactive ingredients, not considering water and pH adjusting agent(s).

In some embodiments, the aqueous pharmaceutical composition herein (e.g., any of the aqueous solution described in [1]-[26] in the Brief Summary section herein) has a total number of three (3) inactive ingredients, not considering water and pH adjusting agent(s).

In any of the embodiments described herein, unless otherwise specified or contrary from context, the aqueous pharmaceutical composition herein (e.g., any of the aqueous solution described in [1]-[26] in the Brief Summary section herein) can be characterized as having the cyclodextrin, the water-soluble antioxidant, and a chelating agent as the inactive ingredients or only inactive ingredients, not considering water and pH adjusting agent(s). Suitable cyclodextrin, water-soluble antioxidant, and chelating agents, and amounts or concentrations are described herein.

Typically, the fosaprepitant and palonosetron, or pharmaceutically acceptable salts thereof, are the only active ingredients in the aqueous pharmaceutical composition herein (e.g., any of the aqueous solution described in [1]-[26] in the Brief Summary section herein). However, in some embodiments, the aqueous pharmaceutical composition herein can comprise additional active ingredients.

In some embodiments, the present disclosure provides an aqueous pharmaceutical composition comprising a ready to use (RTU) aqueous composition of Fosaprepitant or a pharmaceutically acceptable salt thereof, and palonosetron or a pharmaceutically acceptable salt thereof, a cyclodextrin derivative in a pharmaceutically acceptable vehicle, with pharmaceutically acceptable additive and a pH adjusting agent; wherein the composition does not contain polysorbate 80. The amounts or concentrations of each ingredient include any of those described herein.

In some embodiments, the present disclosure provides a stable anti-emetic composition suitable for parenteral injection, the composition comprising: fosaprepitant dimeglumine; sulfobutyl ether β-cyclodextrin; edetate disodium; a pharmaceutically acceptable carrier; and a pH adjusting agent; wherein the composition does not contain polysorbate 80; wherein a weight ratio of the fosaprepitant dimeglumine to the sulfobutyl ether β-cyclodextrin is about 1:10 to about 1:255; and wherein stability of the composition is maintained for at least one month at 40° C. The amounts or concentrations of each ingredient include any of those described herein.

In some embodiments, the present disclosure provides a ready to use aqueous composition of fosaprepitant or a pharmaceutically acceptable salt, such as fosaprepitant dimeglumine, and palonosetron or a pharmaceutically acceptable salt thereof, such as palonosetron hydrochloride, a cyclodextrin derivative, wherein the aqueous compositions have a pH range from about 8 to 12, preferably from about 9 to 11. The amounts or concentrations of each ingredient include any of those described herein.

In some embodiments, the present disclosure provides a ready to use aqueous composition that has (1) the same or substantially the same pH (e.g., within ±0.3 or within ±0.2 units), (2) the same ingredients; and (3) the same or substantially the same (e.g., within ±10% or ±5%) concentrations of the respective ingredients, as those of Formulations 1-7 as described in the Examples section of this application.

In some embodiments, the present disclosure provides a stable ready to use aqueous composition of fosaprepitant comprising fosaprepitant dimeglumine in an amount from about 1.6 mg/mL to 16.4 mg/mL, more preferably from 1.6 mg/mL to 9.8 mg/mL, and palonosetron comprising palonosetron hydrochloride in an amount about 1 μg/mL to 16.7 μg/mL, more preferably from 1 μg/mL to 11 μg/mL, a cyclodextrin derivative selected from the derivatives of β-cyclodextrin, such as sulfobutyl ether β-cyclodextrin, and may also include other formulation stabilizers, such chelating agents and antioxidants, wherein the aqueous compositions have a pH range from about 8 to 12, preferably from about 9 to 11. The amounts or concentrations of each ingredient include any of those described herein.

In some embodiments, the present disclosure provides a stable ready to use aqueous composition of fosaprepitant comprising fosaprepitant dimeglumine, palonosetron hydrochloride, and sulfobutyl ether β-cyclodextrin, wherein the weight ratio of fosaprepitant to sulfobutyl ether β-cyclodextrin is between about 1:10 to 1:255, preferably from about 1:30 to 1:255, wherein the aqueous compositions have a pH range from about 8 to 12, preferably from about 9 to 11. The composition further contains up to 0.1% (w/v) of a chelating agent, such as edetate disodium and up to 2% (w/v) of an antioxidant, such as methionine, wherein the composition is physically and chemically stable for at least 3 months at 40° C. and the chelating agent and antioxidant can be any such chelating agent and antioxidant, respectively, familiar to the skilled in the field. The amounts or concentrations of each ingredient include any of those described herein.

In some embodiments, the present disclosure provides a ready to use aqueous composition comprising fosaprepitant and palonosetron or a pharmaceutically acceptable salts, at least one pharmaceutically acceptable additive in a pharmaceutically acceptable vehicle with a controlled pH, wherein the fosaprepitant and palonosetron composition exhibits excellent physical and chemical stabilities, wherein the said composition on storage for up to 3 months at 25° C. continue to exhibit as a clear and colorless solution without visible precipitates and wherein the said composition on storage generates not more than 3.0% (w/w) drug related degradants. The amounts or concentrations of each ingredient include any of those described herein.

In some embodiments, the present disclosure provides a stable ready to use aqueous composition of fosaprepitant comprising fosaprepitant dimeglumine, combined with palonosetron comprising palonosetron hydrochloride, a sulfobutyl ether β-cyclodextrin, edetate disodium or similar chelating agent, methionine or similar antioxidant, alone or in combination, in water for injection at a controlled pH of approximately 9 to 11. The amounts or concentrations of each ingredient include any of those described herein.

In some embodiments, the present disclosure provides a storage stable, ready to use aqueous composition of fosaprepitant and palonosetron, suitable for parenteral administration prepared following the process of dissolving fosaprepitant or a pharmaceutically acceptable salt in water for injection to form a homogeneous solution. To this solution, a cyclodextrin derivative, such as sulfobutyl ether β-cyclodextrin is added and stirred to yield a clear solution. To the solution formed, palonosetron or pharmaceutically acceptable salt thereof, a chelating agent, such as edetate disodium and an antioxidant, such as methionine, are further added and stirred to completely dissolved, although palonosetron or pharmaceutically acceptable salt thereof and the inactive ingredients can also be added at appropriate steps during the entire process familiar to the skilled in the art. The solution is then adjusted to a target pH using NaOH and/or HCl or other pharmaceutically acceptable pH adjusting agents and brought to the final volume. The final solution is subsequently subjected to sterile filtration, aseptically filled and packed in an appropriate container and closure system. The amounts or concentrations of each ingredient include any of those described herein.

In some embodiments, aqueous compositions of fosaprepitant or its pharmaceutically acceptable salt, such as dimeglumine salt, palonosetron or its pharmaceutically acceptable salt, such as hydrochloride salt, according to the present disclosure comprises a stable aqueous solution of fosaprepitant and palonosetron with a cyclodextrin derivative, such as sulfobutyl ether β-cyclodextrin sodium salt, wherein the amount of fosaprepitant is from about 1.6 mg/mL to 16.4 mg/mL, more preferably from 1.6 mg/mL to 9.8 mg/mL as fosaprepitant dimeglumine, and about 1 μg/mL to 16.7 μg/mL, more preferably from 1 μg/mL to 11 μg/mL as palonosetron hydrochloride, wherein the weight ratio of fosaprepitant to the cyclodextrin derivative is between about 1:10 to 1:255, preferably about 1:30 to 1:255, of fosaprepitant to a cyclodextrin derivative with a solution pH controlled between about 8 to 12, preferably between about 9 to 11. The aqueous pharmaceutical composition herein demonstrates extended storage stability, both physically and chemically, wherein the total degradation products, mainly the active pharmaceutical ingredient, aprepitant, formed should be no more than 10.0% weight/weight (w/w), preferably not more than 5.0% (w/w).

In accordance with one aspect of the present disclosure, the aqueous pharmaceutical composition herein can comprise:
fosaprepitant or a pharmaceutically acceptable salt thereof;
palonosetron or a pharmaceutically acceptable salt thereof;
a dual functional agent of hydrolysis inhibition and solubility enhancement;
a pharmaceutically acceptable vehicle;
one or two inactive pharmaceutical additives;
pH adjusted to between about 9 to 11.
   wherein the relative amount of fosaprepitant and the dual functional agent of hydrolysis inhibition and solubility enhancement are maintained at appropriate ratios, as described herein.

In some embodiments, the present disclosure provides an aqueous pharmaceutical composition comprising (1) fosaprepitant, or a pharmaceutically acceptable salt thereof; (2) palonosetron, or a pharmaceutically acceptable salt thereof; (3) sulfobutyl ether beta-cyclodextrin; (4) a water-soluble antioxidant; and (5) water; wherein the pH of the aqueous pharmaceutical composition ranges from about 9 to about 12. The amounts or concentrations of each ingredient (1)-(5) include any of those described herein.

In some embodiments, the present disclosure provides an aqueous pharmaceutical composition comprising (1) fosaprepitant, or a pharmaceutically acceptable salt thereof; (2) palonosetron, or a pharmaceutically acceptable salt thereof; (3) sulfobutyl ether beta-cyclodextrin; (4) methionine; and (5) water; wherein the pH of the aqueous pharmaceutical composition ranges from about 9 to about 12. The amounts or concentrations of each ingredient (1)-(5) include any of those described herein. In some embodiments, the aqueous pharmaceutical composition is a ready-to-use aqueous solution. In some embodiments, the aqueous pharmaceutical composition is a ready-to-dilute aqueous solution. In some embodiments, the aqueous pharmaceutical composition further comprises a chelating agent, such as EDTA.

In some embodiments, the present disclosure provides an aqueous pharmaceutical composition comprising (1) fosaprepitant, or a pharmaceutically acceptable salt thereof; (2) palonosetron, or a pharmaceutically acceptable salt thereof; (3) sulfobutyl ether beta-cyclodextrin; (4) methionine; and (5) water; wherein the concentration of fosaprepitant in the aqueous pharmaceutical composition ranges from about 1.6 mg/mL to about 16.4 mg/mL, the concentration of palonosetron in the aqueous pharmaceutical composition ranges from about 1 μg/mL to about 16.7 μg/mL, the weight ratio of fosaprepitant to the sulfobutyl ether beta-cyclodextrin ranges from about 1:10 to about 1:255, methionine is present in the aqueous pharmaceutical composition at a concentration ranging from about 0.1 mg/ml to about 30 mg/ml, and the pH of the aqueous pharmaceutical composition ranges from about 9 to about 12. Additional suitable amounts or concentrations of each ingredient (1)-(5) include any of those described herein. In some embodiments, the aqueous pharmaceutical composition is a ready-to-use aqueous solution. In some embodiments, the aqueous pharmaceutical composition is a ready-to-dilute aqueous solution. In some embodiments, the aqueous pharmaceutical composition further comprises a chelating agent, such as EDTA. In some embodiments, the only inactive ingredients in the aqueous pharmaceutical composition are the sulfobutyl ether beta-cyclodextrin, methionine, and the chelating agent (e.g., EDTA). In some embodiments, the fosaprepitant and palonosetron, or pharmaceutically acceptable salts thereof, are the only active ingredients in the aqueous pharmaceutical composition. To be clear, when counting inactive ingredients in the aqueous pharmaceutical compositions herein, water, pH adjusting agents such as HCl or NaOH, or salt formed from adjusting pH, such as NaCl, or components formed during the process of preparation such as meglumine, should not be considered. To further explain, when it is said that the only inactive ingredients in the aqueous pharmaceutical composition are the sulfobutyl ether beta-cyclodextrin, methionine, and the chelating agent (e.g., EDTA), the aqueous pharmaceutical composition can be prepared by mixing the sulfobutyl ether beta-cyclodextrin, methionine, and the chelating agent (e.g., EDTA), and the active ingredients (e.g., fosaprepitant dimeglumine salt and palonosetron hydrochloride salt) in water to form an aqueous mixture, followed by adjusting the pH of the aqueous mixture formed to the recited range (e.g., about 9 to about 12).

In some embodiments, the present disclosure provides an aqueous pharmaceutical composition comprising (1) fosaprepitant, or a pharmaceutically acceptable salt thereof; (2) palonosetron, or a pharmaceutically acceptable salt thereof; (3) sulfobutyl ether beta-cyclodextrin; (4) methionine; (5) EDTA; and (6) water; wherein the concentration of fosaprepitant in the aqueous pharmaceutical composition ranges from about 1.6 mg/mL to about 16.4 mg/mL, the concentration of palonosetron in the aqueous pharmaceutical composition ranges from about 1 μg/mL to about 16.7 μg/mL, the weight ratio of fosaprepitant to the sulfobutyl ether beta-cyclodextrin ranges from about 1:10 to about 1:255, methionine is present in the aqueous pharmaceutical composition at a concentration ranging from about 0.1 mg/ml to about 30 mg/ml, EDTA is present in the aqueous pharmaceutical composition at a concentration ranging from about 0.01 mg/mL to about 10 mg/mL, and the pH of the aqueous pharmaceutical composition ranges from about 9 to about 12. Additional suitable amounts or concentrations of each ingredient (1)-(6) include any of those described herein. In some embodiments, the aqueous pharmaceutical composition is a ready-to-use aqueous solution. In some embodiments, the aqueous pharmaceutical composition is a ready-to-dilute aqueous solution. In some embodiments, the only inactive ingredients in the aqueous pharmaceutical composition are the sulfobutyl ether beta-cyclodextrin, methionine, and EDTA. In some embodiments, the fosaprepitant and palonosetron, or pharmaceutically acceptable salts thereof, are the only active ingredients in the aqueous pharmaceutical composition.

In any of the embodiments described herein, unless otherwise specified or contrary from context, the aqueous pharmaceutical composition herein can be characterized as being storage stable, which may also be referred to herein simply as stable. For example, in any of the embodiments described herein, unless otherwise specified or contrary from context, the aqueous pharmaceutical composition herein (e.g., any of the aqueous solution described in [1]-[26] in the Brief Summary section herein) can be characterized as being free of visible precipitation, i.e., no visible particles in the aqueous pharmaceutical composition are observed. In some embodiments, in any of the embodiments described herein, unless otherwise specified or contrary from context, the aqueous pharmaceutical composition herein (e.g., any of the aqueous solution described in [1]-[26] in the Brief Summary section herein) can be characterized in that, when stored at temperature (25° C.+/−2° C.) for 6 months, the aqueous pharmaceutical composition is considered unchanged physically or chemically, which may be characterized having the same amount of active ingredients (within 3% of the initial amount) and/or having essentially the same appearance (through visual observation, for example, all being clear solution). In some embodiments, in any of the embodiments described herein, unless otherwise specified or contrary from context, the aqueous pharmaceutical composition herein (e.g., any of the aqueous solution described in [1]-[26] in the Brief Summary section herein) can be characterized in that, when stored at temperature (25° C.+/−2° C.) for 6 months, the aqueous pharmaceutical composition is characterized by one or more (e.g., two or more, three or more, four or more, or all) of the following: (1) the amount of aprepitant in the aqueous pharmaceutical composition is less than 5% by weight of the weight of fosaprepitant; (2) the impurity having a relative retention time of 0.65 to fosaprepitant is less than 0.1% by weight of the weight of fosaprepitant; (3) the impurity having a relative retention time of 0.91 to fosaprepitant is less than 0.1% by weight of the weight of fosaprepitant; (4) the impurity having a relative retention time of 2.32 to fosaprepitant is less than 0.1% by weight of the weight of fosaprepitant; (5) the impurity having a relative retention time of 1.06 to palonosetron is less than 0.1% by weight of the weight of palonosetron; and (6) the aqueous solution remains a clear solution free of visible precipitation. Typically, the aqueous pharmaceutical composition can be characterized as satisfying at least one stability profile relating to fosaprepitant (e.g., any one or more of (1)-(4) and/or others described herein), at least one stability profile relating to palonosetron (e.g., (5) and/or others described herein), or a combination thereof, preferably, at least one stability profile relating to fosaprepitant and at least one stability profile relating to palonosetron. In preferred embodiments, the aqueous pharmaceutical composition can be characterized as satisfying the physical stability feature of (6).

In some embodiments, the aqueous pharmaceutical composition herein (e.g., any of the aqueous solution described in [1]-[26] in the Brief Summary section herein) can be characterized in that, when stored at temperature (25° C.+/−2° C.) for 6 months, the aqueous pharmaceutical composition is characterized by 1, 2, 3, or 4 of the following: (1) the amount of aprepitant in the aqueous pharmaceutical composition is less than 5% by weight of the weight of fosaprepitant; (2) the impurity having a relative retention time of 0.65 to fosaprepitant is less than 0.1% by weight of the weight of fosaprepitant; (3) the impurity having a relative retention time of 0.91 to fosaprepitant is less than 0.1% by weight of the weight of fosaprepitant; and (4) the impurity having a relative retention time of 2.32 to fosaprepitant is less than 0.1% by weight of the weight of fosaprepitant. In some embodiments, the aqueous pharmaceutical composition herein (e.g., any of the aqueous solution described in [1]-[26] in the Brief Summary section herein) can be characterized in that, when stored at temperature (25° C.+/−2° C.) for 6 months, the aqueous pharmaceutical composition is characterized in that the combined fosaprepitant related impurities are in an amount of less than 5% (e.g., less than 3%) by weight of the weight of fosaprepitant. In some embodiments, the aqueous pharmaceutical composition herein (e.g., any of the aqueous solution described in [1]-[26] in the Brief Summary section herein) can be characterized in that, when stored at temperature (25° C.+/−2° C.) for 6 months, the aqueous pharmaceutical composition is characterized in that the combined fosaprepitant related impurities, excluding aprepitant, are in an amount of less than 0.3% (e.g., less than 0.2% or less than 0.16%) by weight of the weight of fosaprepitant.

In some embodiments, the aqueous pharmaceutical composition herein (e.g., any of the aqueous solution described in [1]-[26] in the Brief Summary section herein) can be characterized in that, when stored at temperature (25° C.+/−2° C.) for 6 months, the aqueous pharmaceutical composition is characterized in that the impurity having a relative retention time of 1.06 to palonosetron is less than 0.1% by weight of the weight of palonosetron. In some embodiments, the aqueous pharmaceutical composition herein (e.g., any of the aqueous solution described in [1]-[26] in the Brief Summary section herein) can be characterized in that, when stored at temperature (25° C.+/−2° C.) for 6 months, the aqueous pharmaceutical composition is characterized in that no known impurities of palonosetron, Impurities A, B, and C (described herein), is observed.

In some embodiments, the aqueous pharmaceutical composition herein (e.g., any of the aqueous solution described in [1]-[26] in the Brief Summary section herein) can be characterized in that, when stored at temperature (25° C.+/−2° C.) for 6 months, the aqueous solution remains a clear solution free of visible precipitation.

In some embodiments, in any of the embodiments described herein, unless otherwise specified or contrary from context, the aqueous pharmaceutical composition herein (e.g., any of the aqueous solution described in [1]-[26] in the Brief Summary section herein) can be characterized in that, when stored at temperature (25° C.+/−2° C.) for 6 months, (1) the amount of aprepitant in the aqueous solution is less than 5% by weight of the weight of fosaprepitant; (2) the impurity having a relative retention time of 1.06 to palonosetron is less than 0.1% by weight of the weight of palonosetron; and (3) the aqueous solution remains a clear solution free of visible precipitation.

In any of the embodiments described herein, unless otherwise specified or contrary from context, the aqueous pharmaceutical composition herein (e.g., any of the aqueous solution described in [1]-[26] in the Brief Summary section herein) is suitable for pharmaceutical use, such as suitable for parenteral injection, in particular, bolus intravenous injection or intravenous infusion.

In any of the embodiments described herein, unless otherwise specified or contrary from context, the aqueous pharmaceutical composition herein (e.g., any of the aqueous solution described in [1]-[26] in the Brief Summary section herein) can be an aqueous solution suitable for pharmaceutical use, such as suitable for parenteral injection, in particular, bolus intravenous injection or intravenous infusion.

In any of the embodiments described herein, unless otherwise specified or contrary from context, the aqueous pharmaceutical composition herein (e.g., any of the aqueous solution described in [1]-[26] in the Brief Summary section herein) can be a ready-to-use formulation, which can be used directly without further handling, such as reconstitution and/or dilution.

In any of the embodiments described herein, unless otherwise specified or contrary from context, the aqueous pharmaceutical composition herein (e.g., any of the aqueous solution described in [1]-[26] in the Brief Summary section herein) can be in a single-dose dosage form, such as packaged in an ampoule, a vial, a cartridge, a pre-filled syringe, or an intravenous bag.

In any of the embodiments described herein, unless otherwise specified or contrary from context, the aqueous pharmaceutical composition herein (e.g., any of the aqueous solution described in [1]-[26] in the Brief Summary section herein) can be in a multi-dose dosage form.

### Methods of Preparing Aqueous Pharmaceutical Compositions

In some embodiments, the present disclosure also provides a method of preparing the aqueous pharmaceutical compositions herein.

In a broad aspect, the methods of preparing the aqueous pharmaceutical compositions herein typically include mixing the ingredients in the desired amount/concentration described herein in water. For example, in some embodiments, the ingredients and the respective amount in the aqueous pharmaceutical composition herein can be any of those described in [1]-[26] in the Brief Summary section herein.

In some particular embodiments, the method of preparation herein comprises (a) dissolving the fosaprepitant or pharmaceutically acceptable salt in water to form a first solution; (b) mixing the cyclodextrin with the first solution to provide a second solution; and (c) mixing the palonosetron or pharmaceutically acceptable salt thereof, water-soluble antioxidant, and optional other ingredients to the second solution, thereby forming the aqueous pharmaceutical composition. In some embodiments, the method further comprises adjusting the pH of the aqueous pharmaceutical composition to about 9 to about 12 (e.g., about 9 to about 11, such as about 9.5, about 10, about 10.5, about 11, or any ranges or values between the recited values, such as about 10-11). It should be noted that the cyclodextrin, the palonosetron or pharmaceutically acceptable salt thereof, water-soluble antioxidant, and optional other ingredients, can be first dissolved in water before mixing with the first or second solution, as appropriate. For example, in some embodiments, the cyclodextrin is dissolved in water before mixing with the first solution. In some embodiments, the cyclodextrin is added to the first solution as a solid.

In some embodiments, the present disclosure provides a method of preparing a stable anti-emetic composition suitable for parenteral injection, the method comprising the steps of dissolving fosaprepitant or a pharmaceutically acceptable salt thereof in water forming a first solution; mixing palonosetron or a pharmaceutically acceptable salt thereof in the first solution to form a second solution; adding a cyclodextrin derivative to the second solution to form a third solution; and adjusting the pH of the third solution. It should be noted that the cyclodextrin derivative and the palonosetron or pharmaceutically acceptable salt thereof, can be first dissolved in water before mixing with the first or second solution, as appropriate. For example, in some embodiments, the cyclodextrin derivative is dissolved in water before being added to the second solution. In some embodiments, the cyclodextrin derivative is added to the second solution as a solid.

It is an object of the present embodiments to provide a ready to use aqueous composition of fosaprepitant or a pharmaceutically acceptable salt thereof, combined with palonosetron or a pharmaceutically acceptable salt thereof, at least one pharmaceutically acceptable additive in a pharmaceutically acceptable vehicle with a controlled pH, wherein the fosaprepitant composition exhibits excellent both physical and chemical stabilities. The amounts or concentrations of each ingredient include any of those described herein. In some embodiments, the method further comprises adjusting the pH of the aqueous pharmaceutical composition to about 9 to about 12 (e.g., about 9 to about 11, such as about 9.5, about 10, about 10.5, about 11, or any ranges or values between the recited values, such as about 10-11).

In some particular embodiments, the method of preparation herein comprises (a) dissolving fosaprepitant dimeglumine salt in water to form a first solution; (b) mixing sulfobutyl ether beta-cyclodextrin sodium salt with the first solution to provide a second solution; (c) mixing palonosetron hydrochloride salt, methionine, and EDTA disodium salt with the second solution to form a third solution; and (d) adjusting the pH of the third solution to about 9 to about 12 (e.g., about 9 to about 11, such as about 9.5, about 10, about 10.5, about 11, or any ranges or values between the recited values, such as about 10-11), wherein, based on the volume of the final aqueous pharmaceutical composition:
the fosaprepitant dimeglumine salt in (a) is about 1.6 mg/mL to about 16.4 mg/mL (e.g., about 1.6 mg/mL to about 9.8 mg/mL);
the sulfobutyl ether beta-cyclodextrin sodium salt in (b) is about 50 mg/ml to about 300 mg/ml (such as about 50 mg/ml, about 60 mg/ml, about 70 mg/ml, about 80 mg/ml, about 90 mg/ml, about 100 mg/ml, about 120 mg/ml, about 140 mg/ml, about 160 mg/ml, about 180 mg/ml, about 200 mg/ml, about 250 mg/ml, about 300 mg/ml, or any ranges or values between the recited values, such as about 60-200 mg/ml, about 80-180 mg/ml, about 64 mg/ml, etc.);
the palonosetron hydrochloride salt in (c) is about 1 μg/mL to about 16.7 μg/mL (e.g., about 1 μg/mL to about 11 μg/mL);
the methionine in (c) is about 0.1 mg/ml to about 30 mg/ml (such as about 0.5 mg/ml, about 1 mg/ml, about 1.5 mg/ml, about 2 mg/ml, about 2.5 mg/ml, about 5 mg/ml, about 10 mg/ml, about 15 mg/ml, about 20 mg/ml, about 30 mg/ml, or any ranges or values between the recited values, such as about 1.5-2.5 mg/ml, about 2-5 mg/ml, etc.); and
the EDTA disodium salt in (c) is about 0.01 mg/mL to about 10 mg/mL (preferably, about 0.1 mg/mL to about 1 mg/mL).

It should be noted that the sulfobutyl ether beta-cyclodextrin sodium salt, the palonosetron hydrochloride salt, methionine, and EDTA disodium salt, can be first dissolved in water before mixing with the first or second solution, as appropriate. For example, in some embodiments, the sulfobutyl ether beta-cyclodextrin sodium salt is dissolved in water before mixing with the first solution. In some embodiments, the sulfobutyl ether beta-cyclodextrin sodium salt is added to the first solution as a solid. In some embodiments, the weight ratio of fosaprepitant dimeglumine salt to the sulfobutyl ether beta-cyclodextrin sodium salt ranges from about 1:13 to about 1:255, such as about 1:20, about 1:25, about 1:30, about 1:35, about 1:40, about 1:50, about 1:75, about 1:100, about 1:150, about 1:200, about 1:250, or any ranges or values between the recited values, such as about 1:20 to about 1:50, about 1:30 to about 1:40; about 1:25 to about 1:100, etc. Those skilled in the art would appreciate that when a different salt form of any of the ingredients is used, the amount of such different salt form can be adjusted so as to arrive at the same concentration for the respective ingredient as described above.

In some embodiments, the method of preparation herein can include a sterilization step.

In some embodiments, the method of preparation herein can further include packaging or sealing the aqueous pharmaceutical composition in a suitable container, such as an ampoule, a vial, a cartridge, a pre-filled syringe, or an intravenous bag. In some embodiments, prior to sealing the suitable container, inert gas such as N2 or Ar can be used to replace the air in the container or otherwise reduce the oxygen content in the headspace of the container.

In some embodiments, the method of preparation herein is for preparing a single-dose dosage form.

In some embodiments, the method of preparation herein is for preparing a multi-dose dosage form.

In any of the embodiments according to the method of preparation herein, unless otherwise specified or contrary from context, the method is for preparing a ready-to-use formulation, which does not include lyophilizing the aqueous pharmaceutical composition herein.

However, in some embodiments, the method can also comprise lyophilizing the aqueous pharmaceutical composition herein to form a lyophilized powder, which can be reconstituted prior to use.

It should be noted that the aqueous pharmaceutical composition or aqueous solution prepared by any of the methods herein is also a novel composition of the present disclosure. Typically, the aqueous pharmaceutical composition or aqueous solution prepared by the methods herein is storage stable, e.g., as described herein.

### Methods of Treatment

The aqueous pharmaceutical compositions herein have various utilities.

In some embodiments, the aqueous pharmaceutical compositions herein can be used for treating or preventing any disease or disorder for which administering fosaprepitant and/or palonosetron has been known to be beneficial. Such diseases or disorders include any of those approved by a regulatory agency, such as the U.S. Food and Drug Administration and the alike.

For example, in some embodiments, the present disclosure provides a method of treating or preventing nausea and/or vomiting in a subject in need thereof, the method comprising administering to the subject an effective amount of any of the aqueous pharmaceutical composition herein (e.g., any of the aqueous solution described in [1]-[26] in the Brief Summary section herein). In some embodiments, the nausea and/or vomiting is a chemotherapy induced nausea and/or vomiting. In some embodiments, the nausea and/or vomiting is induced by a radiation therapy. In some embodiments, the nausea and/or vomiting is induced by a surgical procedure.

In some embodiments, the method herein is for the prevention of acute and delayed nausea and vomiting associated with initial and repeat courses of highly emetogenic cancer chemotherapy (HEC) including high-dose cisplatin.

In some embodiments, the method herein is for the prevention of delayed nausea and vomiting associated with initial and repeat courses of moderately emetogenic cancer chemotherapy (MEC).

In some embodiments, the method herein is for the prevention of postoperative nausea and/or vomiting.

In some embodiments, the method is for treating an adult patient.

In some embodiments, the method is for treating a pediatric patient, such as patients 6 months of age or older, such as age 12-17.

Typically, the method of treatment herein comprises administering the aqueous pharmaceutical composition herein intravenously, such as bolus intravenous injection or intravenous infusion.

The aqueous pharmaceutical compositions herein can be used in a combination therapy with additional active ingredients. In some embodiments, in the methods herein, the subject is only treated with fosaprepitant and palonosetron as active ingredients for or treating or preventing the nausea and/or vomiting. When used in a combination therapy, one or more of the aqueous pharmaceutical compositions herein can be administered with an additional therapy (e.g., an additional antiemesis therapy) either concurrently or sequentially in any order.

The dosing regimen of using the aqueous pharmaceutical compositions herein is not particularly limited. For example, in some embodiments, one or more of the aqueous pharmaceutical compositions herein can be administered to a subject in need according to a treatment regimen described in the U.S. FDA approved labels for fosaprepitant injection.

### Alternative Exemplary Embodiments

In some embodiments, the present disclosure also provides the following alternative exemplary enumerated embodiments 1-20.

1. A composition comprising fosaprepitant and palonosetron, wherein said composition is an aqueous liquid solution.

2. The composition of embodiment 1, wherein the composition further comprises cyclodextrin.

3. The composition of embodiment 2, wherein the aqueous liquid solution is adjusted to a desired target pH using NaOH and/or HCl solutions.

4. The composition of embodiment 3, wherein the desired target pH is between about 8.5 and 11.

5. The composition of embodiment 4, wherein the composition is made by first dissolving fosaprepitant and the cyclodextrin in two separate parts of 60-80% water to form two separate parts of clear solutions, and then the two separate parts of clear solutions are mixed together to form a mixed solution, and subsequently adding palonosetron to the mixed solution.

6. The composition of embodiment 5, wherein the palonosetron that is added to the mixed solution is in an aqueous solution.

7. The composition of embodiment 5, wherein the desired target pH is between about 9.6 to 11.

8. The composition of embodiment 5, wherein the composition is made at about 25° C.

9. The composition of embodiment 5, wherein the composition is filtered through a 0.22 m sterile filter and filled into pre-sterilized containers under pressure.

10. The composition of embodiment 9, wherein the composition further comprises a dual functional agent of hydrolysis inhibition and solubility enhancement, a pharmaceutically acceptable vehicle, and another pharmaceutical additive.

11. A method of making a composition comprising fosaprepitant, cyclodextrin, and palonosetron, wherein the composition is an aqueous solution, said method comprising dissolving fosaprepitant and cyclodextrin in 60-80% water to form a clear solution, and subsequently adding palonosetron to the clear solution.

12. The method of embodiment 11, wherein the fosaprepitant and the cyclodextrin are mixed in two separate parts of 60-80% water, and then added together.

13. The method of embodiment 11, wherein a pH is adjusted to a pH between about 8.5 and 11 by addition of a solution of NaOH and/or HCl.

14. The method of embodiment 13, wherein the composition is filtered through a 0.22 m sterile filter.

15. The method of embodiment 14, further comprising adding the composition to one or more pre-sterilized containers wherein the containers are under vacuum.

16. The method of embodiment 15, wherein the composition further comprises a dual functional agent of hydrolysis inhibition and solubility enhancement, and a pharmaceutically acceptable vehicle.

17. A method of inhibiting or treating nausea in an individual, said method comprising administering to said individual in need thereof an aqueous solution composition comprising fosaprepitant, cyclodextrin, and palonosetron.

18. The method of embodiment 17, wherein the aqueous solution composition further comprises a pharmaceutically acceptable excipient, diluent, or surfactant.

19. The method of embodiment 17, wherein the aqueous solution composition is administered parenterally.

20. The method of embodiment 19, wherein a dose of the fosaprepitant and palonosetron is sufficient to alleviate symptoms of nausea in a patient in need thereof.

### Definition

Unless defined otherwise, all technical and scientific terms used herein have the same meaning as commonly understood by one of ordinary skill in the art to which the disclosure pertains.

It is understood that wherever embodiments, are described herein with the language “comprising” otherwise analogous embodiments, described in terms of “containing” “consisting of” and/or “consisting essentially of” are also provided. However, when used in the claims as transitional phrases, each should be interpreted separately and in the appropriate legal and factual context (e.g., in claims, the transitional phrase “comprising” (or including or containing) is considered more of an open-ended phrase while “consisting of” is more exclusive and “consisting essentially of” achieves a middle ground).

As used herein, the singular form “a”, “an”, and “the”, includes plural references unless it is expressly stated or is unambiguously clear from the context that such is not intended.

As used herein, the term “about” modifying an amount related to the invention refers to variation in the numerical quantity that can occur, for example, through routine testing and handling; through inadvertent error in such testing and handling; through differences in the manufacture, source, or purity of ingredients employed in the invention; and the like. As used herein, “about” a specific value also includes the specific value, for example, about 10% includes 10%. Whether or not modified by the term “about”, the claims include equivalents of the recited quantities. In one embodiment, the term “about” means within 20% of the reported numerical value. In one embodiment, the term “about” means within 10% of the reported numerical value. In one embodiment, the term “about” means within 5% of the reported numerical value.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Headings and subheadings are used for convenience and/or formal compliance only, do not limit the subject technology, and are not referred to in connection with the interpretation of the description of the subject technology. Features described under one heading or one subheading of the subject disclosure may be combined, in various embodiments, with features described under other headings or subheadings. Further it is not necessarily the case that all features under a single heading or a single subheading are used together in embodiments.

The term “solution” means a homogeneous liquid phase containing two or more Substances, where the two Substances are intimately combined so as to behave physically as a single phase.

As used herein, a pharmaceutical composition that is “free of” of an ingredient should be understood that the aqueous pharmaceutical composition does not include the ingredient in a detectable amount. Typically, such pharmaceutical composition can be prepared without using the ingredient. For example, when it is said that a composition is free of a surfactant, the composition does not include any detectable amount of surfactant, which can be prepared by not using a surfactant. Other expressions should be understood similarly.

As used herein, a pharmaceutical composition that is “substantially free of” of an ingredient should be understood that the aqueous pharmaceutical composition does not include the ingredient in an amount greater than 10% (w/v), more preferably, the aqueous pharmaceutical composition does not include the ingredient in an amount greater than 5% (w/v), preferably, no greater than 3% (w/v), or no greater than 1% (w/v).

The term “subject” (alternatively referred to herein as “patient”) as used herein, refers to an animal, preferably a mammal, most preferably a human, who has been the object of treatment, observation or experiment.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated.

As used herein, the terms “prevent,” “preventing,” “prevention,” and the like refer to reducing the likelihood for developing, delaying or eliminating the appearance of a disease or condition, delaying or eliminating the onset of symptoms of a disease or condition, slowing, halting, or reversing the progression of a disease or condition, or any combination thereof by administering a drug before the onset of the disease or condition.

### EXAMPLES

For illustration purpose only, the following are selected examples. The ingredients used for the examples are generally commercially available. For example,

SBECD: Betadex sulfobutyl ether sodium, USP, was obtained from vendor in Shandong China. L-Methionine USP, EP, JP, was obtained from Sigma-Aldrich, Cat. No. 64319. Excipients used for the examples are pharmaceutical grades, or in compliance with USP.

### Example 1. Preparation of Formulations and Stability Studies

The following general procedures were used to prepare the exemplary formulations herein:
weigh water of about 60-80% of the batch size to be prepared into an appropriate compounding container and bring water to the desired temperature;
add batch required amount of fosaprepitant dimeglumine and mix until completely dissolved;
followed by adding the batch required amount of sulfobutyl ether beta-cyclodextrin sodium, in increments if necessary, under continuous mixing and mix until all cyclodextrin derivative is completely dissolved to form a homogeneous solution;
the composition required amount of palonosetron or its pharmaceutically acceptable salt is further added and mixed until complete dissolution.

Alternatively, the exemplary formulations can be prepared using the following general procedures:
first dissolving fosaprepitant and sulfobutyl ether beta-cyclodextrin sodium in two separate parts of the 60-80% water to form two separate parts of clear solutions;
mixing together the two solutions to form a fosaprepitant and sulfobutyl ether beta-cyclodextrin clear homogenous solution;
adding required amount of palonosetron;
and any other ingredients into the composition during any steps shown above;
adjusting the solution to a desired target pH using NaOH and/or HCl solutions;
adding additional water is used to bring the total volume to the intended batch size;

The Final product solution obtained for the exemplary formulations can be further filtered through a 0.22 μm sterile filter and filled into pre-sterilized container closure system under protection.

Formulations 1-11 prepared were tested for storage stabilities at 25° C. HPLC was used to analyze fosaprepitant, palonosetron, and relevant impurities. The level of impurities is expressed as % w/w, in relation to the initial amount of fosaprepitant or palonosetron, respectively, unless otherwise specified.

The HPLC conditions used for analysis of fosaprepitant and its related substances (or impurities) and palonosetron and its related substances (impurities) are two separate gradient methods that are briefly summarized as the following:

TABLE 1
HPLC Methods Conditions for Analyzing Fosaprepitant and Palonosetron
and their respective related substances (or Impurities)

| HPLC | Fosaprepitant and | Palonosetron and Related |
|---|---|---|
| condition | Related Substances | Substances |
| Column | Column, XBridge C₁₈, | Column, XBridge C₁₈, 250 |
| | 250 mm × 4.6 mm, | mm × 4.6 mm, |
| | 5 μm, Waters (PN | 3.5 μm, Waters (PN 186003943) |
| | 186003117) | or equivalent |
| | or equivalent | |
| Mobile phase | Mobile phase A: an | Mobile phase A: a potassium |
| | ammonium | hexafluorophosphate-sodium |
| | dihydrogen phosphate | perchlorate buffer system |
| | buffer solution | Mobile phase B: 100% |
| | Mobile phase B: 100% | Acetonitrile |
| | Acetonitrile | |
| UV Detection | 215 nm | 243 nm |
| Flow rate | 1.0 mL/min | 0.8 mL/min |
| Injection | 10 μL | 160 μL |
| volume | | |

### Example 2. Preparation of Formulations 1-11

Formulations 1-7 were prepared according to the general procedures described in Example 1; details of the ingredients are shown in the tables below. Formulations 8-11 only contains one of the two active ingredients, i.e., either fosaprepitant or palonosetron, prepared similar to the procedure described in Example 1: Formulation 1

Formulation 1

| Component | Concentration (mg/mL) |
|---|---|
| Fosaprepitant dimeglumine | 4.906 |
| Palonosetron hydrochloride | 0.005 |
| Sulfobutyl ether β-cyclodextrin sodium | 160 |
| Edetate disodium | 0.108 |
| NaOH/HCl | q.s. pH 11 |
| Water for injection | q.s. 1.0 mL |

Formulation 2

| Component | Concentration (mg/mL) |
|---|---|
| Fosaprepitant dimeglumine | 4.906 |
| Palonosetron hydrochloride | 0.005 |
| Sulfobutyl ether β-cyclodextrin sodium | 160 |
| Edetate disodium | 0.108 |
| Methionine | 20 |
| NaOH/HCl | q.s. pH 10.5 |
| Water for injection | q.s. 1.0 mL |

Formulation 3

| Component | Concentration (mg/mL) |
|---|---|
| Fosaprepitant dimeglumine | 4.906 |
| Palonosetron hydrochloride | 0.005 |
| Sulfobutyl ether β-cyclodextrin sodium | 160 |
| Edetate disodium | 0.108 |
| Methionine | 2 |
| NaOH/HCl | q.s. pH 10.5 |
| Water for injection | q.s. 1.0 mL |

Formulation 4

| Component | Concentration (mg/mL) |
|---|---|
| Fosaprepitant dimeglumine | 16.353 |
| Palonosetron hydrochloride | 0.0167 |
| Sulfobutyl ether β-cyclodextrin sodium | 160 |
| Edetate disodium | 0.108 |
| Methionine | 10 |
| NaOH/HCl | q.s. pH 10.5 |
| Water for injection | q.s. 1.0 mL |

Formulation 5

| Component | Concentration (mg/mL) |
|---|---|
| Fosaprepitant dimeglumine | 4.906 |
| Palonosetron hydrochloride | 0.005 |
| Sulfobutyl ether β-cyclodextrin sodium | 80 |
| Edetate disodium | 0.108 |
| Methionine | 10 |
| NaOH/HCl | q.s. pH 10.5 |
| Water for injection | q.s. 1.0 mL |

Formulation 6

| Component | Concentration (mg/mL) |
|---|---|
| Fosaprepitant dimeglumine | 4.906 |
| Palonosetron hydrochloride | 0.005 |
| Sulfobutyl ether β-cyclodextrin sodium | 160 |
| Edetate disodium | 0.108 |
| Methionine | 10 |
| NaOH/HCl | q.s. pH 9.5 |
| Water for injection | q.s. 1.0 mL |

Formulation 7

| Component | Concentration (mg/mL) |
|---|---|
| Fosaprepitant dimeglumine | 4.906 |
| Palonosetron hydrochloride | 0.005 |
| Sulfobutyl ether β-cyclodextrin sodium | 160 |
| Methionine | 10 |
| NaOH/HCl | q.s. pH 10.5 |
| Water for injection | q.s. 1.0 mL |

Formulation 8

| Component | Concentration (mg/mL) |
|---|---|
| Palonosetron hydrochloride | 0.005 |
| Sulfobutyl ether β-cyclodextrin sodium | 160 |
| Sodium metabisulfite | 2 |
| NaOH/HCl | q.s. pH 10.5 |
| Water for injection | q.s. 1.0 mL |

Formulation 9

| Component | Concentration (mg/mL) |
|---|---|
| Palonosetron hydrochloride | 0.005 |
| Sulfobutyl ether β-cyclodextrin sodium | 160 |
| Methionine | 2 |
| NaOH/HCl | q.s. pH 10.5 |
| Water for injection | q.s. 1.0 mL |

Formulation 10

| Component | Concentration (mg/mL) |
|---|---|
| Fosaprepitant Dimeglumine | 4.906 |
| Sulfobutyl ether β-cyclodextrin sodium | 64 |
| Edetate Disodium | 0.376 |
| NaOH/HCl | q.s. pH 8.5 |
| Water for injection | q.s. 1.0 mL |

Formulation 11

| Component | Concentration (mg/mL) |
|---|---|
| Fosaprepitant Dimeglumine | 4.906 |
| Sulfobutyl ether β-cyclodextrin sodium | 64 |
| Edetate Disodium | 0.376 |
| NaOH/HCl | q.s. pH 10 |
| Water for injection | q.s. 1.0 mL |

### Example 3. Stabilities of Formulations 1-11

Selected Formulations 1-11 were tested for stabilities at 25° C. The results are shown in Tables 1-4 below:

TABLE 1
Stability of Fosaprepitant (FSPT)* in Composition Over Time at 25° C.

| | | | | FSPT | Impurities/degradants (% w/w) | | | | |
|---|---|---|---|---|---|---|---|---|---|
| | | Time | | % of | | | RRT | | |
| | Storage | Point | | Initial | RRT | RRT | 2.22 | RRT | Total |
| Batch Info | Condition | (month) | Appearance | Content | 0.65 | 0.91 | (APPT) ** | 2.32 | Impurities |
| Formulation | 25° C. | Initial | clear | 102.14 | ND | 0.024 | 0.211 | 0.044 | 0.279 |
| 5 | | 1 | clear | 101.62 | 0.083 | 0.038 | 0.558 | 0.068 | 0.747 |
| | | 3 | clear | 101.12 | 0.053 | 0.041 | 1.470 | 0.090 | 1.654 |
| | | 6 | clear | 99.18 | 0.046 | 0.034 | 2.406 | 0.065 | 2.551 |

*RRT in Table 1 refers to the relative retention time to fosaprepitant.
** APPT refers to aprepitant.

TABLE 2
Stability of Palonosetron (PLNS) in Composition Over Time at 25° C.

| | | | | Impurities/degradants (% w/w) | | |
|---|---|---|---|---|---|---|
| | | Time | PLNS | RRT* | RRT | |
| | Storage | Point | % of Initial | 0.95 | 1.06 | Total |
| Batch Info | Condition | (month) | Content | (Imp C) | (Imp A) | Impurities |
| Formulation | 25° C. | Initial | 100.00% | ND | ND | 0.000 |
| 5 | | 1 | 98.73% | ND | ND | 0.000 |
| | | 3 | 100.88% | ND | ND | 0.000 |
| | | 6 | 100.29% | ND | ND | 0.000 |

*RRT in Table 2 refers to the relative retention time to palonosetron.

TABLE 3
Difference in Stabilization Effect of Methionine and Sodium Metabisulfite on Palonosetron at pH 10

| | | | | | Impurities/degradants (% Theoretical) | | | |
|---|---|---|---|---|---|---|---|---|
| | | | | API | RRT* | RRT | RRT | |
| | Storage | Time Point | | Content | 1.06 | 0.97 | 0.95 | Total |
| Batch Info | Condition | (month) | Appearance | (mg/mL) | (Imp A) | (Imp B) | (Imp C) | Impurities |
| Formulation 8 | N/A | Initial | Clear solution | 0.00496 | ND | ND | ND | ND |
| Methionine | 25° C. | 1 | Clear solution | 0.00499 | ND | ND | ND | ND |
| 2 mg/ml | | | | | | | | |
| | | 3 | Clear solution | 0.00490 | ND | ND | ND | ND |
| | | 6 | Clear solution | 0.00471 | ND | ND | ND | ND |
| | | | | | Impurities/degradants (% Theoretical) | | | |
| | | | | API | RRT | RRT | RRT | |
| | Storage | Time Point | | Content | 1.06 | 0.97 | 0.95 | Total |
| Batch Inf | Condition | (month) | Appearance | (mg/mL) | (Imp A) | (Imp B) | (Imp C) | Impurities |
| Formulation 9 | N/A | Initial | Clear solution | 0.00515 | ND | ND | ND | ND |
| Sodium | 25° C. | 1 | Clear solution | 0.00528 | 0.237 | ND | ND | 0.237 |
| metabisulfite | | | | | | | | |
| 2 mg/ml | | | | | | | | |
| | | 3 | Clear solution | 0.00509 | 1.023 | ND | ND | 1.023 |
| | | 6 | Clear solution | 0.00492 | 2.363 | ND | ND | 2.363 |

*RRT in Table 3 refers to the relative retention time to palonosetron.

TABLE 4
Chemical and Physical Stability of Fosaprepitant in Aqueous
Formulations of Different pH (Formulations 10 and 11)

| Formulation | Aprepitant Formed (% w/w) over Time (month) at 25° C. | | | | | | |
|---|---|---|---|---|---|---|---|
| pH | 0 | 0.25 | 0.5 | 1 | 2 | 3 | 6 |
| 8.5 | 0.196 | ND | 0.759 | 1.213 | 2.450 | 3.586 | Precipitation |
| 10 | 0.178 | 0.258 | 0.368 | 0.569 | 0.988 | 1.262 | 2.540 |

The known impurities or related compounds of Palonosetron are:
Palonosetron Related Impurity A (Imp A): (3aS)-1-Oxo-2-[(3S)-quinuclidin-3-yl]-2,3,3a,4,5,6-hexahydro-1H-benzo[de]isoquinoline 1-oxide.
Palonosetron Related Impurity B(Imp B): (3S)-3-(1-Oxo-5,6-dihydro-1H-benzo[de]isoquinolin-2(4H1)-yl)quinuclidine 1-oxide.
Palonosetron Related Impurity C (Imp C): (3aS)-2-[(3R)-Quinuclidin-3-yl]-2,3,3a,4,5,6-hexahydro-1H-benzo[de]isoquinolin-1-one hydrochloride.

The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of” or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

1. An aqueous solution suitable for parenteral injection comprising:
(1) fosaprepitant, or a pharmaceutically acceptable salt thereof;
(2) palonosetron, or a pharmaceutically acceptable salt thereof;
(3) a cyclodextrin;
(4) a water-soluble antioxidant; and
(5) water;
wherein the aqueous solution comprises water in an amount of greater than 50% by weight;
wherein the concentration of fosaprepitant in the aqueous solution ranges from about 1.6 mg/mL to about 16.4 mg/mL;
the concentration of palonosetron in the aqueous solution ranges from about 1 μg/mL to about 16.7 μg/mL;
the weight ratio of fosaprepitant to the cyclodextrin ranges from about 1:10 to about 1:255; and
the pH of the aqueous solution ranges from about 9 to about 12,
wherein the concentration of fosaprepitant or weight ratio of fosaprepitant to the cyclodextrin is calculated based on equivalent weight to fosaprepitant dimeglumine salt;
and the concentration of palonosetron is calculated based on equivalent weight to palonosetron hydrochloride salt.

2. The aqueous solution of claim 1, wherein the water-soluble antioxidant is a sulfur containing antioxidant, such as an amino acid, such as methionine, or monothioglycerol, or sodium metabisulfite, etc.

3. (canceled)

4. The aqueous solution of claim 1, wherein the water-soluble antioxidant is present in the aqueous solution at a concentration ranging from about 0.1 mg/ml to about 30 mg/ml.

5. (canceled)

6. The aqueous solution of claim 1, wherein the cyclodextrin is sulfobutyl ether beta-cyclodextrin, preferably, a sulfobutyl ether-β-cyclodextrin having an average degree of substitution of about 3-10, such as about 4.5-7.5 or about 6-7.1, or a sodium salt thereof.

7. The aqueous solution of claim 1, wherein (i) the cyclodextrin is present in the aqueous solution at a concentration ranging from about 50 mg/ml to about 300 mg/ml; (ii) the concentration of fosaprepitant in the aqueous solution ranges from about 1.6 mg/mL to about 9.8 mg/mL; and/or (iii) the concentration of palonosetron in the aqueous solution ranges from about 1 μg/mL to about 11 μg/mL.

8. (canceled)

9. (canceled)

10. The aqueous solution of claim 1, wherein (i) the weight ratio of fosaprepitant to the cyclodextrin ranges from about 1:13 to about 1:255; and/or the pH of the aqueous solution is about 9 to about 11.

11. (canceled)

12. The aqueous solution of claim 1, wherein (i) the aqueous solution is free of or substantially free of a surfactant; (ii) the aqueous solution is free of or substantially free of a water-soluble polymer or protein; and/or (iii) the aqueous solution is free of or substantially free of a buffer agent.

13. (canceled)

14. (canceled)

15. (canceled)

16. (canceled)

17. The aqueous solution of claim 1, wherein the aqueous solution further comprises a chelating agent.

18. The aqueous solution of claim 17, wherein chelating agent is EDTA.

19. The aqueous solution of claim 1, wherein the aqueous solution has a total number of two to five (2-5) inactive ingredients, not considering water and pH adjusting agent(s).

20. The aqueous solution of claim 19, wherein the inactive ingredients in the aqueous solution, not considering water and pH adjusting agent(s), comprise or consist of the cyclodextrin, the water-soluble antioxidant, and a chelating agent.

21. The aqueous solution of claim 1, wherein the fosaprepitant and palonosetron, or pharmaceutically acceptable salts thereof, are the only active ingredients in the aqueous solution.

22. The aqueous solution of claim 1, which is free of visible precipitation.

23. The aqueous solution of claim 1, characterized in that when stored at room temperature (25° C.+/−2° C.) for 6 months, the aqueous solution is characterized by one or more of the following: (1) the amount of aprepitant in the aqueous solution is less than 5% by weight of the weight of fosaprepitant; (2) the impurity having a relative retention time of 0.65 to fosaprepitant is less than 0.1% by weight of the weight of fosaprepitant; (3) the impurity having a relative retention time of 0.91 to fosaprepitant is less than 0.1% by weight of the weight of fosaprepitant; (4) the impurity having a relative retention time of 2.32 to fosaprepitant is less than 0.1% by weight of the weight of fosaprepitant; (5) the impurity having a relative retention time of 1.06 to palonosetron is less than 0.1% by weight of the weight of palonosetron; and (6) the aqueous solution remains a clear solution free of visible precipitation.

24. The aqueous solution of claim 1, which is a ready-to-use formulation.

25. (canceled)

26. (canceled)

27. A lyophilized powder made from the aqueous solution of claim 1.

28. A method of preparing the aqueous solution of claim 1, the method comprising:
(a) dissolving the fosaprepitant or pharmaceutically acceptable salt in water to form a first solution;
(b) mixing the cyclodextrin with the first solution to provide a second solution;
(c) mixing the palonosetron or pharmaceutically acceptable salt thereof, water-soluble antioxidant, and optional other ingredients to the second solution to form a third solution, and
(d) optionally adjusting the pH of the third solution, thereby forming the aqueous solution.

29. A method of treating nausea and/or vomiting, comprising administering to a subject in need the aqueous solution of claim 1.

30. The method of claim 29, wherein the administering is by parenteral injection.

31. The method of claim 29, wherein the nausea and/or vomiting is a chemotherapy, a radiation therapy, or a surgical procedure induced nausea and/or vomiting.
